(19) United States
(12) Patent Application Publication
(10) Pub. No.: US 2013/0040874 A1
(43) Pub. Date: Feb. 14, 2013

(54) Branched Hydroxyalkyl Polyoxylene Glycol Ethers

(71) Applicants: Hans-Christian Raths, Monheim (DE); Sabine Both, Neuss (DE); Frederic Bauer, Dusseldorf (DE); Manfred Weuthen, Langenfeld (DE)

(72) Inventors: Hans-Christian Raths, Monheim (DE); Sabine Both, Neuss (DE); Frederic Bauer, Dusseldorf (DE); Manfred Weuthen, Langenfeld (DE)

(73) Assignee: Cognis IP Management GmbH, Dusseldorf (DE)

(21) Appl. No.: 13/643,176

(22) PCT Filed: Jan. 29, 2011
(86) PCT No.: PCT/EP2011/000412

§ 371 Date: Oct. 24, 2012

(30) Foreign Application Priority Data
Apr. 27, 2010 (EP) EP10004428

(51) Int. Cl. C07C 43/13; C11D 3/20

(52) U.S. Cl. 510/506; 568/623

(57) ABSTRACT

Certain branched hydroxyalkyl polyoxylene glycol ethers exhibit excellent characteristics as defoamers or foam-suppressing additives or as foam regulators for aqueous media that contain foaming agents, even at temperatures of 25° C. and below. Therefore, said branched hydroxyalkyl polyoxylene glycol ethers can be used in detergents and cleaning agents.

The present invention relates to selected branched surfactants of the hydroxyalkyl polyoxylene glycol ether type and to the use thereof in washing or cleaning compositions, in particular for defoaming, foam control or foam suppression of aqueous compositions which comprise foaming components.

Aqueous washing and cleaning compositions often comprise substances which lead to undesired foam formation under the use conditions. These substances are often anionic surfactants, e.g. of the alkylbenzenesulfonate type or of the linear alkyl sulfate type and of the fatty alcohol polyglycol ether sulfate type, or certain non-ionic surfactants, such as alkyl(oligo)glycosides. In addition, however, various constituents of the soilings to be cleaned off or substances located in the application system and/or introduced into the application system with surface-active properties can form foams and/or stabilize the foam tendency in an application and thus bring about or intensify the formation of foams. By way of example, mention may be made of substances and substance groups such as e.g. proteins, polymers, saponified fats, wood fibers and ingredients thereof which form foams. In order to counteract undesired foaming, therefore, either a) foam-suppressing additives are used, i.e. substances which prevent the formation of foams, or b) antifoams are used, i.e. agents which are able to destroy existing foams. Foam regulators are known as a third category c), i.e. substances which do not completely suppress foam formation, but are able to limit the foam volume to a certain degree. Details relating to this can be found in the literature, e.g. in N. D. Denkov, Mechanisms of Foam Destruction by Oil-based Antifoams, Langmuir 2004, 20, 9463-9505, or in the TEGEWA puplication “Foam” by the “Surface-active substances” project group from 2007. Foams are stabilized by the solid and homogeneous surfactant film on the liquid—It is therefore the task of a defoamer to destroy this film, and/or of a foam-suppressing additive to prevent its formation.

The foam-suppressing or defoaming additives used are usually hydrophobic oils (e.g. mineral oil, paraffin oils, fatty alcohols and esters thereof, fatty acids and silicone oils) and a combination thereof with finely distributed hydrophobic solids. However, certain non-ionic surfactants can also exhibit a defoaming and/or foam-suppressing effect. For example, hydroxyalkyl polyoxylene glycol ethers are used here. These are known compounds which can be obtained e.g. by reacting a 1,2-epoxyalkane with an alkylated fatty alcohol. Here, DE 19738866 A1 is used as an example of the prior art. EP 0 299 360 A2, for example discloses such compounds and their use as foam-suppressing additives. Although the broad Markush formula of EP 0 299 360 A2 also includes branched derivatives, only compounds with linear alkyl radicals are specifically disclosed in the specification to the person skilled in the art. EP 1 299 103 A1 discloses hydroxyl polyoxylene glycol ethers and their use in cleaning compositions, the use of branched alcohols also being described in the description for the production, without specific compounds of this type or properties thereof appearing to have been disclosed.

Although many substances are already known which are suitable as defoamers and/or foam-suppressing additives in aqueous compositions, new substances are also sought. Since current washing and cleaning compositions which develop their effect at the lowest possible use temperatures (preferably less than 30° C. and in particular at temperatures of less than 25° C.) are demanded by consumers and industrial and/or commercial users, defoamers and/or foam-suppressing additives or foam regulators are also sought which either exhibit the same effectiveness at these low temperatures as known compounds at higher use temperatures, or exhibit better effectiveness than prior art substances at the same temperatures.

It has now been found that certain selected hydroxyalkyl polyoxylene glycol ethers exhibit these desired properties.

A first subject matter of the present invention therefore relates to hydroxyalkyl polyoxylene glycol ethers of the general formula (I)

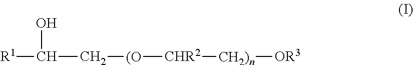

in which R¹ is a saturated linear alkyl radical having 6 to 18 carbon atoms, R² is a hydrogen atom or a methyl radical, where the radical R³ is a mono- or polybranched saturated alkyl radical having 5 to 24 carbon atoms, and the index n is integers or fractions from 2 to 20.

Hydroxyalkyl polyoxylene glycol ethers are known in principle. In EP 299 360 A2, which has already been cited above, references to branched radicals R¹ or R³ can also be found in the description without compounds according to the description of the present formula (I) appearing to have been specifically and unambiguously predescribed.

The teaching of the present invention is now based on the surprising finding that the defoaming and/or foam-suppressing effect of the hydroxyalkyl polyglycol ethers is retained at low temperatures upon exclusively varying the polyoxyglycol-terminal radical R³, whereas the purely linear derivatives disclosed specifically from the prior art lose their effectiveness.

The compounds of the general formula (I) are usually prepared by reacting alkoxylated branched fatty alcohols with aryl oxiranes in the presence of alkaline catalysts. The preparation processes are known per se and are also disclosed in EP 0 299 360 A2. The branched radicals R³ in the general formula (I) thus originate from the alcohol which has been alkoxylated prior to the reaction with the oxirane.

Suitable branched alcohols are, for example, the four isopentanols, in particular isoamyl alcohol (3-methyl-butan-1-ol), and further suitable examples are 2-methylpentyl alcohol, 2-methylhexyl alcohol, 2-methylheptyl alcohol, 2-methyloctyl alcohol, 2-methylnonyl alcohol, 2-methyldecyl alcohol, and also 2-ethylpropyl alcohol, 2-ethylbutyl alcohol, 2-ethylpentyl alcohol, 2-ethylhexyl alcohol, 2-ethylheptyl alcohol, 2-ethyloctyl alcohol, 2-ethylnonyl alcohol, 2-ethyldecyl alcohol. 3-Methyl- or 3-ethylpentanol, -hexanol, -heptanol, -octanol and -nonanol are suitable branched alcohols. Isononanols or isodecanols are also suitable branched alcohols for the purposes of the present teaching. Besides the monobranched alcohols, polybranched alcohols having 6 to 12 carbon atoms may also be suitable. The alcohols can also be used in the form of any desired mixtures with one another. Also all branched alcohols having 6 to 12 carbon atoms obtained by Guerbet reaction, and mixtures thereof, may be particularly suitable.

Particular preference is given to 2-ethylhexyl alcohol or isooctanol. 2-Ethylhexanol is a synthetic alcohol which is based on propene as raw material. Propene is reacted with CO and hydrogen to give butanal, which is then converted to 2-ethyl-3-hydroxyethanol by aldol condensation. Elimination of water and catalytic hydrogenation give 2-ethylhexanol.

Isooctanol can also be obtained by so-called oxosynthesis from olefins, carbon monoxide (CO) and hydrogen. This synthesis is also called hydroformylation. The aldehydes formed during the hydroformylation are then hydrogenated. Various primary isoalcohols are obtained depending on the olefin used. Thus, a isoheptene mixture produces, as a result of hydroformylation and subsequent hydrogenation, a mixture consisting mostly of 3,3-, 3,5- and 4,5-dimethyl-1-hexanol alongside 3- and 5-methyl-1-heptanol. 6-Methyl-1-heptanol is also a widespread industrially available isooctanol. Further particularly suitable branched alcohols are isononanol and isodecanol; here too, mixtures are present. This is because isononanol is a mixture of branched primary isononanols with a large fraction of 3,5,5-trimethylhexan-1-ol. In the case of isodecanol, a mixture of isomeric trimethylheptanols alongside 3,5-dimethyloctan-1-ol is present.

The hydroxyalkyl polyoxylene glycol ethers of the general formula (I) can contain ethylene oxide groups, propylene oxide groups or both ethylene oxide and propylene oxide groups together, in which case these can then be present either blockwise or randomized in the molecule. If propylene oxide groups are present, these conform to the general formula (Ia):

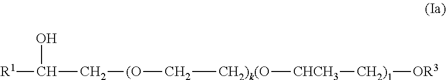

Where the radicals R¹ and R³ have the meaning given above and the index k is an integer or fraction from 1 to 16, and 1 is an integer or fraction from 1 to 4, preferably 1 to 3 and in particular 1 to 2, with the proviso that k+1=n. The value of the index k in the general formula (Ia) is then 2 to 16, preferably 4 to 12 and in particular 6 to 10.

Preferred compounds of this type comprise e.g. 2 mol of propylene oxide and 10 mol of ethylene oxide per mole of the hydroxyalkyl polyoxylene glycol ether, or 14 mol of ethylene oxide and 3 mol of propylene oxide per mole of the hydroxyalkyl polyoxylene glycol ether.

The order of the groups (O—CH₂—CH₂)ₖ(O—CHCH₃—CH₂)₁ in the formula (Ia) does not stipulate a sequence; rather, this formula encompasses or stands for all conceivable groups (O—CH₂—CH₂)ₖ and (O—CHCH₃—CH₂)₁ distributed block-wise and randomized within the Markush formula (Ia). In particular, those compounds of the formula (Ia) in which the fraction of propylene oxide groups (O—CHCH₃—CH₂) is lower than the fraction of ethylene oxide groups (O—CH₂CH₂) are selected here. It is also possible to use e.g. purely ethoxylated and purely propoxylated compounds of the formula (I) alongside one another. However, particularly preferred compounds of the general formula (I) always contain ethylene oxide; preferably, they contain only ethylene oxide groups, and are thus free from propylene oxide groups.

The degree of alkoxylation, characterized in the general formula (I) (i.e. the fraction of alkoxide groups, i.e. ethylene oxide and/or propylene oxide groups) by the index n is preferably from 3 to 20, or from 5 to 15 and in particular from 6 to 11.

In a particularly preferred embodiment, those molecules of the formula (I) in which R² is a hydrogen atom (these are exclusively ethoxylated derivatives), and the radical R³ is a 2-ethylhexyl radical are selected. Hydroxyalkyl polyoxylene glycol ethers according to the general formula (I) in which R³ is a 2-ethylhexyl radical and n is an integer or fraction from 6 to 10 and in particular from 7 to 9, and R² is a linear alkyl radical having 6 to 12 carbon atoms are likewise preferred.

The radical R¹ originates from the oxirane which is used in the reaction. Preferably, linear saturated alkyl radicals having 6 to 12, preferably 6 to 10 and in particular 8 to 10, carbon atoms are selected for R¹ in the formula (I).

The compounds according to the general formula (I) are suitable for defoaming and also as foam-suppressing additives or as foam regulators for aqueous systems, compositions or solutions, emulsions or dispersions which comprise foaming and/or foam-stabilizing components. As well as foaming surface-active substances, such as surfactants, other substances which are primarily introduced from soilings, such as proteins, polymers, saponified fats, wood fibers and ingredients thereof, and saponines can also form and/or stabilize foams and therefore act as a foaming component, foam stabilizer and/or foam former.

The compounds of the general formula (I) can also be used as foam regulators. Suitable foaming surfactants are in particular anionic surfactants, here preferably alkyl sulfates, alkyl ether sulfates, alkylsulfonates and/or alkylbenzenesulfonates. Further foam formers to be mentioned would be foaming non-ionic surfactants such as, for example, alkyl(oligo)glycosides and alkoxylated, preferably ethoxylated, fatty alcohols, but also amine oxides, betaines, fatty acid amides and alkoxylated derivatives thereof and other cationic surfactants.

Alkyl sulfates and/or alkenyl sulfates, which are often also referred to as fatty alcohol sulfates, are to be understood as meaning the sulfation products of primary alcohols which conform to the formula (II),

R⁴O—SO₃X (II)

in which R⁴ is a linear or branched, aliphatic alkyl and/or alkenyl radical having 6 to 22, preferably 12 to 18, carbon atoms and X is an alkali metal and/or alkaline earth metal, ammonium, alkylammonium, alkanol-ammonium or glucammonium. Typical examples of alkylsulfates which can be used within the context of the invention are the sulfation products of caproic alcohol, capryl alcohol, capric alcohol, 2-ethylhexyl alcohol, lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol and erucyl alcohol, and technical-grade mixtures thereof which are obtained by high-pressure hydrogenation of technical-grade methyl ester fractions or aldehydes from the Roelen oxo synthesis. The sulfation products can preferably be used in the form of their alkali metal salts and in particular their sodium salts. Particular preference is given to alkyl sulfates based on C_{16/18}-tallow fatty alcohols and vegetable fatty alcohols of comparable carbon chain distribution in the form of their sodium salts.

Alkyl ether sulfates (“ether sulfates”) are known anionic surfactants which are produced industrially by SO₃— or chlorosulfonic acid (CSA)-sulfation of fatty alcohol or oxo alcohol polyglycol ethers and subsequent neutralization. Within the context of the invention, suitable ether sulfates are those which conform to the formula (III),

R⁵O—(CH₂CH₂O)ₘSO₃X (III)

in which R⁵ is a linear or branched alkyl and/or alkenyl radical having 6 to 22 carbon atoms, n is numbers from 1 to 10 and X is an alkali metal and/or alkaline earth metal, ammonium, alkylammonium, alkanolammonium or glucammonium. Typical examples are the sulfates of addition products of, on average, 1 to 10 mol and in particular 2 to 5 mol, of ethylene oxide onto caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and technical-grade mixtures thereof in the form of their sodium and/or magnesium salts. The ether sulfates can here have either a conventional homolog distribution or a narrow homolog distribution. Particular preference is given to the use of ether sulfates based on adducts of, on average, 2 to 3 mol of ethylene oxide onto technical-grade C_{12/14} or C_{12/18}-coconut fatty alcohol fractions in the form of their sodium and/or magnesium salts.

Alkylbenzenesulfonates preferably conform to the formula R—Ph—SO₃X in which R is a branched, but preferably linear, alkyl radical having 10 to 18 carbon atoms, Ph is a phenyl radical and X is an alkali metal and/or alkaline earth metal, ammonium, alkylammonium, alkanolammonium or glucammonium. Preference is given to using cumenesulfonate, xylenesulfonate, toluene-sulfonate, dodecylbenzenesulfonates, tetradecylbenzene-sulfonates, hexadecylbenzenesulfonates, and technical-grade mixtures thereof, e.g. in the form of the sodium salts.

Alkyl(oligo)glycosides are likewise known. Alkyl and alkenyl oligoglycosides are known non-ionic surfactants which conform to the formula (IV)

R⁶O-[G]ₚ (IV)

in which R⁶ is an alkyl and/or alkenyl radical having 4 to 22 carbon atoms, G is a sugar radical having 5 or 6 carbon atoms and p is numbers from 1 to 10. They can be obtained by the relevant processes of preparative organic chemistry. The alkyl and/or alkenyl oligoglycosides can be derived from aldoses or ketoses having 5 or 6 carbon atoms, preferably glucose. The preferred alkyl and/or alkenyl oligoglycosides are thus alkyl and/or alkenyl oligoglucosides. The index number p in the general formula indicates the degree of oligomerization (DP), i.e. the distribution of mono- and oligoglycosides, and is a number between 1 and 10. Whereas p in a given compound must always be an integer and here in particular can assume the values p=1 to 6, the value p for a certain alkyl oligoglycoside is an analytically determined calculated parameter which in most cases is a fraction. Preferably, alkyl and/or alkenyl oligoglycosides with an average degree of oligomerization p of from 1.1 to 3.0 are used. From an application point of view, preference is given to those alkyl and/or alkenyl oligoglycosides whose degree of oligomerization is less than/equal to 1.7 and is in particular between 1.2 and 1.6, preferably 1.2 to 1.4. The alkyl or alkenyl radical R⁶ can be derived from primary alcohols having 4 to 11, preferably 8 to 10, carbon atoms. Typical examples are butanol, caproic alcohol, capryl alcohol, capric alcohol and undecyl alcohol, and technical-grade mixtures thereof, as are obtained for example in the hydrogenation of technical-grade fatty acid methyl esters or in the course of the hydrogenation of aldehydes from the Roelen oxo synthesis. Preference is given to alkyl oligoglucosides of chain length C₈-C₁₀ (DP=1 to 3), which are produced as forerunner in the distillative separation of technical-grade C₈-C₁₈-coconut fatty alcohol and can be contaminated with a fraction of less than 6% by weight C₁₂-alcohol, and also alkyl oligoglucosides based on technical-grade C_{9/11}-oxo alcohols (DP=1 to 3). The alkyl or alkenyl radical R⁶ can also be derived from primary alcohols having 12 to 22, preferably 12 to 14, carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical-grade mixtures thereof, which can be obtained as described above. Preference is given to alkyl oligoglucosides based on hydrogenated C_{12/14} coconut alcohol with a DP of 1 to 3.

The effectiveness of the compounds of the general formula (I) in the defoaming or as foam suppressants or as foam-controlling additive is particularly pronounced for aqueous systems, emulsions or solutions which comprise alkyl(oligo)glycosides, and therefore the use here is preferred.

Fatty alcohol ethoxylates conform to the general formula (V)

R⁷—(OC₂H₄)₂—OH (V)

in which R⁷ is linear or branched alkyl and/or alkenyl radicals having 8 to 22 carbon atoms and z is a number from 1 to 30 and preferably from 1 to 15, and in particular from 1 to 10 or 1 to 5. Typical examples are the adducts of, on average, 1 to 20 mol onto caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and also technical-grade mixtures thereof which are produced e.g. in the high-pressure hydrogenation of technical-grade methyl esters based on fats and oils or aldehydes from the Roelen oxo synthesis, and as monomer fraction in the dimerization of unsaturated fatty alcohols. Preference is given to adducts of from 10 to 40 mol of ethylene oxide onto technical-grade fatty alcohols having 12 to 18 carbon atoms, such as, for example, coconut, palm, palm kernel or preferably tallow fatty alcohol. Particularly preferred fatty alcohol ethoxylates are based on tallow alcohol which have been ethoxylated with 5 to 25 and preferably 7 to 15 mol of ethylene oxide per mole of alcohol.

The compounds of the general formula (I) are characterized in this connection in that they exhibit their foam-suppressing or defoaming or foam-controlling effect at lower temperatures than e.g. the linear isomers or other agents in the prior art such as silicone defoamers. The effect arises even at temperatures of less than 30° C., in particular temperatures of less than/equal to 25° C. and even at 20° C. and lower temperatures. Furthermore, the compounds of the general formula (I) can be combined and formulated with a large number of other surfactants and other ingredients of washing and cleaning compositions. However, the compositions according to the invention do not have, as has been observed for conventional defoaming additives, such as e.g. hydrophobic oils (e.g. mineral oil, paraffin oils, fatty alcohols and esters thereof, fatty acids and silicone oils), an adverse influence or effect on other cleaning components or on the cleaning result or the cleaning performance. The addition of the compounds of the general formula (I) even unexpectedly leads to an improvement in the cleaning performance in cleaning applications being observed.

The present teaching also encompasses a method for defoaming aqueous surfactant systems (which also includes aqueous emulsions or dispersions), preferably of solutions, where compounds according to the general formula (I) are added to the aqueous surfactant solution in amounts of at least 25 ppm, preferably of at least 50 and in particular of at least 100 ppm (mg/kg of surfactant solution) at temperatures of less than or equal to 25° C. The addition can also take place in portions. It is also possible to initially introduce an aqueous solution comprising the compounds of the formula (I), and then to bring this into contact with the medium to be defoamed and/or to add this.

The compounds of the general formula (I) can thus advantageously be used as additive in order to find use e.g. in existing or newly developing formulations of washing and cleaning compositions for defoaming, as foam suppressant or as foam regulator.

The compounds of the formula (I) furthermore exhibit particularly advantageous properties: for example, with silicone defoamers—as a rule these are silica particles hydromodified with silicone oils—there is no lasting effect, i.e. defoamer must be added over and over in order to avoid new foam formation. The use of the hydroxyalkyl polyoxylene glycol ethers according to the invention as per the general formula (I) leads to a lasting defoaming effect when added just once. Consequently, the amount of defoamer substances is considerably reduced since the amount of active substance does not have to be increased compared with the silicone defoamers. Moreover, the substances according to the present teaching act more quickly compared to other liquid defoamers, a property which is advantageous for many applications.

Moreover, the compounds of the general formula (I) are also able to effectively suppress and/or avoid foams which are formed by alkyl(oligo)glycosides. A property which other defoamers do not have.

The compounds of the general formula (I) can be used in this connection for defoaming and/or as foam-suppressing additives or as foam regulators for all types of aqueous solutions, but also for all types of emulsions (in particular of the W/O and O/W type) or dispersions. These may be e.g. wash liquors or cleaning composition solutions for which only a small amount of foam, if any, should develop.

The hydroxyl polyoxylene glycol ethers of the general formula (I) can be used particularly preferably for cleaning compositions in the sector of industrial and institutional cleaning of hard surfaces, e.g. in the cleaning of bottles made of glass, plastic or metal. Cleaning compositions of this type can advantageously also be used in a very wide variety of spray cleaning applications or so-called CIP (cleaning in place) cleaning applications which require defoaming, foam-suppressing or foam-controlling additives in the application, as e.g. in the cleaning of tanks (e.g. for storing liquid foods which comprise foam formers, as in the case of beer production). A further field of application are foam-controlled products for the surface care of floor coverings using manual, semiautomatic or automatic cleaning appliances.

By reducing the temperature of the aqueous surfactant solution, it is possible for example to reduce the energy input during cleaning compared with conventional defoamers, or it is possible to reduce the amount used at the same temperature.

The present invention further provides washing, rinsing and cleaning compositions which comprise compounds of the general formula (I), preferably in amounts of from 0.001 to 10% by weight, based on the total weight of the compositions, and in particular from 0.01 to 2% by weight. These washing and cleaning compositions can be solid, liquid or gel-like and, besides the compounds of the general formula (I), they also comprise further characteristic ingredients, such as surfactants, bleaches and bleach activators, builder substances, complexing agents, hydrotopes, non-aqueous solvents, polymers, colorants and dyes, inorganic and/or organic pH regulators, preservatives, biocides and/or thickeners.

### EXAMPLES

Preparation of the Hydroxyl Polyoxylene Glycol Ethers:

2-Ethylhexyl alcohol or n-octanol were reacted in each case in the presence of sodium methoxide as catalyst with ethylene oxide at 160° C. to give the ethoxylated alcohol with 9 mol of ethylene oxide per mole of alcohol, and then neutralized. The ethoxylated alcohol from the first step was then reacted in a second reaction step at 160-180° C. in the presence of sodium methoxide as catalyst with a C₁₀-alkyl epoxide to give the desired 2-ethylhexyl-9-EO hydroxydecyl ether (E1) or isooctanol-9EO hydroxydecyl ether (V1), and neutralized.

Application-Related Investigations:

The compounds E1 and V1 were in each case investigated as to their suitability as flame-controlling and flame-suppressing agents or as defoamers:

The measurement of the defoaming effect was carried out in accordance with the DIN method 14371:2004 in a circulation test method.

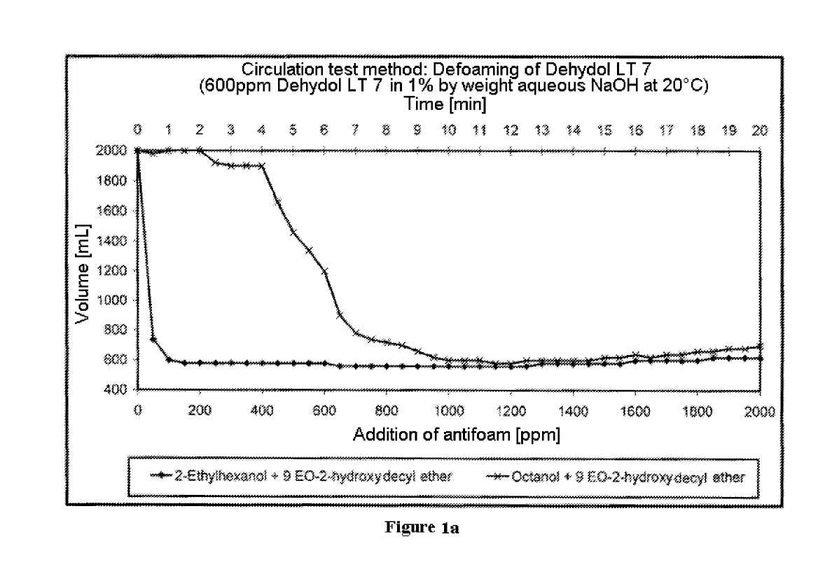

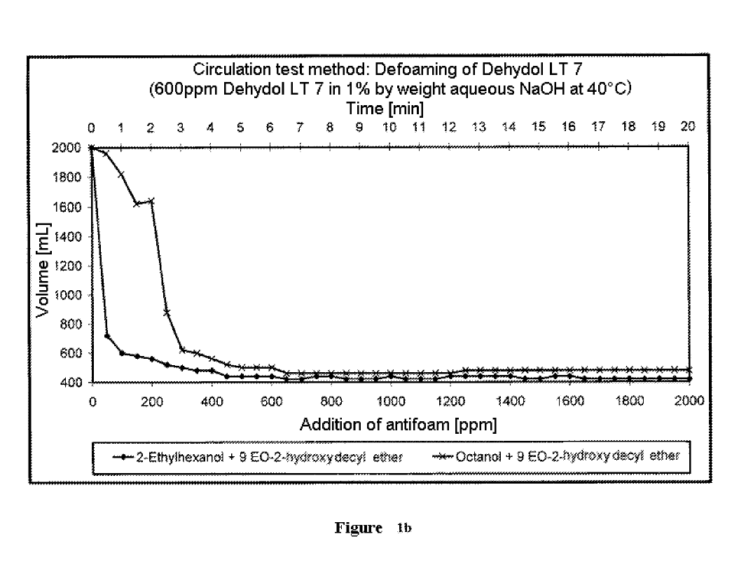

At 20° C. and at 40° C., 500 ml of a 1% by weight aqueous NaOH solution were admixed with 600 ppm of a foaming non-ionic surfactant of the fatty alcohol ethoxylate type (Dehydol® LT 7 from Cognis), and a foam with an active foam volume of 2000 ml was produced. Various amounts of the compounds E1 or V1 were then added to this foam for the purposes of defoaming. The results are shown in figure la (20° C.) and FIG. 1b (40° C.)

It is found that the compound E1 according to the invention in each case exhibits a defoaming effect, even at a relatively low dose, compared to the substance V1.

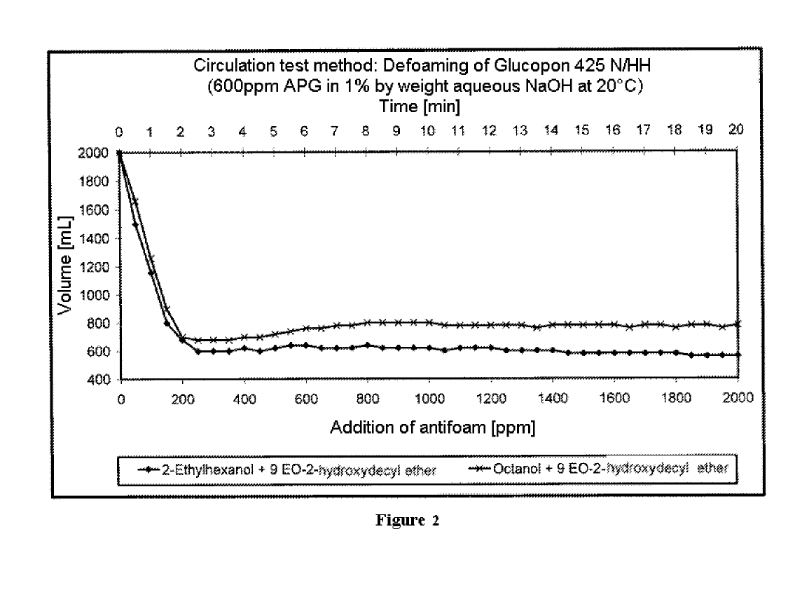

In a further test, the procedure was as above, but instead of the Dehydol® LT 7, an alkyl(oligo)glycoside (Glucopon® 425 N/HH from Cognis) was used as the surfactant. Again, the substances E1 and V1 were added and the defoaming effect was measured as a function of the amount added. The results can be found in FIG. 2. Here too, the composition El according to the invention works better than the comparison substance V1.

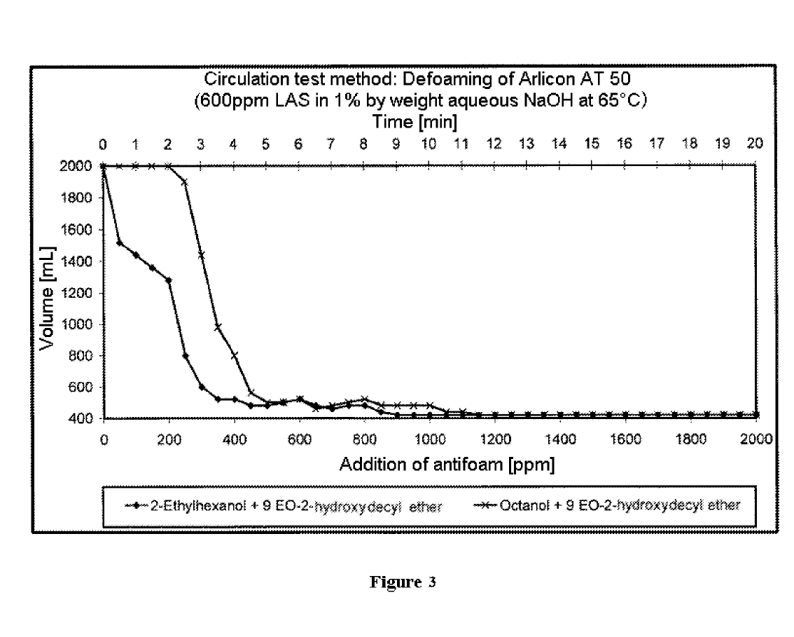

In a further test, which was carried out analogously to the above experiments, the foaming surfactant used was an anionic surfactant of the alkylbenzenesulfonate type (Arlicon® AT 50). The measuring temperature this time was 65° C. The results of the measurement can be found in FIG. 3. Again, the branched product E1 according to the invention exhibits a better effectiveness than the linear comparison product V1.

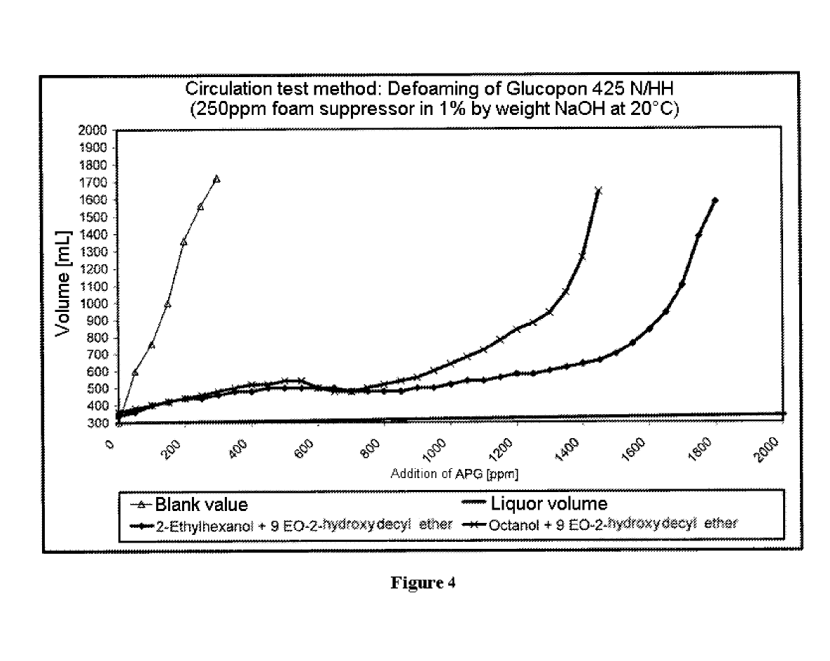

To measure the foam-suppressing effect, by means of a circulation test method and at 20° C. firstly 250 ppm of El were added to 400 ml of an aqueous NaOH solution (1% by weight), and in a second experiment 250 ppm of V1 were added. Then, every 30 seconds, in each case in steps of 50 ppm, the highly foaming alkyl(oligo)glycoside Glucopon® 425 N/HH was metered in, and the foam development was measured. The results can be found in FIG. 4. E1 exhibits a better foam suppression than V1.

Furthermore, the cleaning ability of the compound E1 according to the invention compared with other defoaming surfactants of the prior art was measured. For this, in each case 1% by weight of the surfactant (active substance content) in distilled water were tested at 25° C. in a Gardner test on PVC with a standard soiling (IPP 83/21). For the comparison, a propoxylated fatty alcohol (V2) and a silicone emulsion (V3) were used alongside E1. E1 achieved a cleaning performance of 75%, V2 38% and V3 45%.

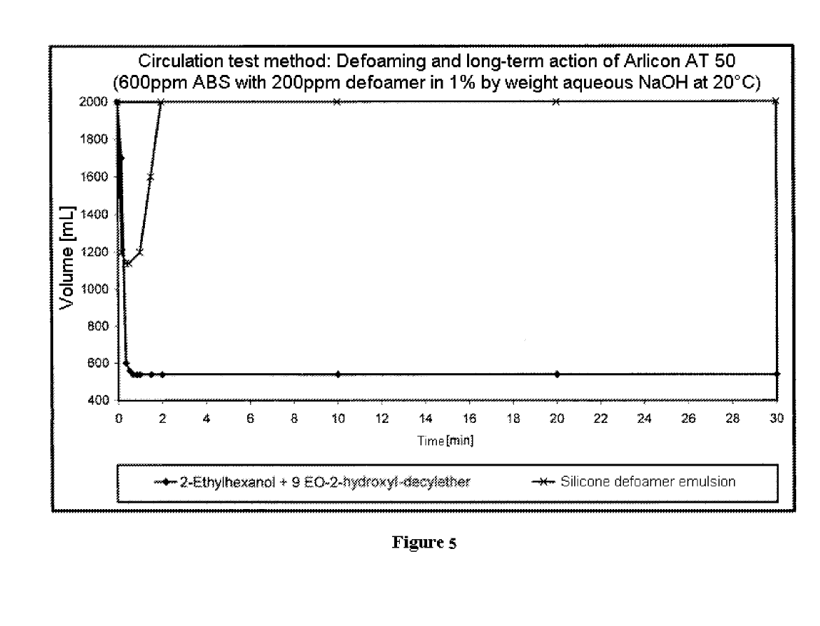

In a further test, the compound E1 was compared with a standard commercial silicone defoamer (TEGO® Antifoam 1488—Evonik). The foam former used was Arlicon® AT 50. As is evident from FIG. 5, E1 defoams more effectively—moreover, the effect is also retained for a long period, whereas the silicone defoamer is not able to develop a lasting foam-controlling effect.

1. A hydroxyalkyl polyoxylene glycol ether of the general formula (I) 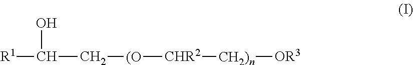
wherein R¹ is a saturated linear alkyl radical having 6 to 18 carbon atoms, R² is a hydrogen atom or a methyl radical, R³ is a mono- or polybranched saturated alkyl radical having 5 to 24 carbon atoms, and n is integers or fractions from 2 to 20.

2. The hydroxyalkyl polyoxylene glycol ether as claimed in claim 1, wherein R² is a hydrogen atom and the radical R³ is a 2-ethylhexyl radical or an isooctanol radical.

3. The hydroxyalkyl polyoxylene glycol ether as claimed in claim 1, wherein R¹ is a saturated linear alkyl radical having 6 to 12 carbon atoms.

4. The hydroxyalkyl polyoxylene glycol ether as claimed in claim 1, wherein n in the general formula (I) is even numbers or fractions from 3 to 20.

5. The hydroxyalkyl polyoxylene glycol ether as claimed in claim 1, wherein, in the general formula (I), R³ is a 2-ethylhexyl radical and n is an even number or fraction from 6 to 10 and R¹ is a linear alkyl radical having 6 to 12 carbon atoms.

6. The hydroxyalkyl polyoxylene glycol ether as claimed in claim 1, wherein R² in the general formula (I) is a hydrogen atom.

7. A method for controlling or suppressing foam formation of an aqueous composition, the method comprising contacting an aqueous composition comprising foam formers with an antifoam or foam-controlling or foam-suppressing additive, wherein the additive comprises one or more of the hydroxyalkyl polyoxylene glycol ethers of claim 1.

8. The method claimed in claim 7, wherein the additive produces an anti-foam, foam-controlling or foam-suppressing effect at temperatures of less than/equal to 25° C.

9. A washing, rinsing or cleaning composition comprising hydroxyalkyl polyoxylene glycol ethers as claimed in claim 1.

10. A method for defoaming aqueous compositions comprising foam formers, the method comprising adding to the aqueous composition one or more compounds according to claim 1 in an amount of at least 25 ppm, at a temperature of less than/equal to 25° C.

11. The hydroxyalkyl polyoxylene glycol ether as claimed in claim 2, wherein R¹ is a saturated linear alkyl radical having 6 to 12 carbon atoms.

12. The hydroxyalkyl polyoxylene glycol ether as claimed in claim 2, wherein n in the general formula (I) is even numbers or fractions from 3 to 20.

13. The hydroxyalkyl polyoxylene glycol ether as claimed in claim 3, wherein R¹ is a saturated linear alkyl radical having 6 to 10 carbon atoms.

14. The hydroxyalkyl polyoxylene glycol ether as claimed in claim 3, wherein n in the general formula (I) is even numbers or fractions from 3 to 20.

15. The hydroxyalkyl polyoxylene glycol ether as claimed in claim 4, wherein the index n in the general formula (I) is even numbers or fractions from 5 to 15.

16. The hydroxyalkyl polyoxylene glycol ether as claimed in claim 5, wherein, in the general formula (I), R³ is a 2-ethylhexyl radical and n is an even number or fraction from 7 to 9 and R¹ is a linear alkyl radical having 6 to 12 carbon atoms.

17. The method claimed in claim 8, wherein the defoaming foam-controlling or foam-suppressing effect arises even at temperatures of less than/equal to 20° C.

18. The method claimed in claim 10, wherein the compound according to claim 1 is added at a temperature of less than/equal to 20° C.

19. The hydroxyalkyl polyoxylene glycol ether as claimed in claim 13, wherein R¹ is a saturated linear alkyl radical having 8 to 10 carbon atoms.

20. The hydroxyalkyl polyoxylene glycol ether as claimed in claim 15, wherein the index n in the general formula (I) is even numbers or fractions from 6 to 11.
